(19) United States
(12) Patent Application Publication
(10) Pub. No.: US 2013/0178724 A1
(43) Pub. Date: Jul. 11, 2013

(54) APPARATUS AND METHOD FOR PREDICTING A PARAMETER IN THE BLOOD STREAM OF A SUBJECT

(71) Applicants: Choon Meng Ting, Singapore (SG); Joon Hock Yeo, Singapore (SG); Xiqin Zhang, Singapore (SG)

(72) Inventors: Choon Meng Ting, Singapore (SG); Joon Hock Yeo, Singapore (SG); Xiqin Zhang, Singapore (SG)

(73) Assignee: GLUCOSTATS SYSTEM PTE LTD, Singapore (SG)

(21) Appl. No.: 13/809,045

(22) PCT Filed: Jul. 7, 2011
(86) PCT No.: PCT/SG2011/000242

§ 371 Date: Mar. 25, 2013

(30) Foreign Application Priority Data
Jul. 8, 2010 (SG) 201004978-1

(51) Int. Cl. A61B 5/1455; A61B 5/145

(52) U.S. Cl. CPC A61B 5/1455; A61B 5/14532
USPC 600/316; 600/479

(57) ABSTRACT

An apparatus and method for predicting a parameter in the blood stream of a subject is disclosed. The apparatus includes a laser diode source arranged to emit light of at least two different wavelengths; a first optical receiver arranged to receive incident light of the two different wavelengths where the subject is not present; a second optical receiver arranged to receive transmitted or diffuse reflected light of the two different wavelengths when a desired part of the subject is present and a processor for calculating the ratio of the intensity of the received transmitted or diffuse reflected light to incident light for each of the at least two different wavelengths to provide an indication of the parameter in the blood stream of the subject. The apparatus and method are particularly suited for measuring HbA1c in an individual.

### FIELD OF THE INVENTION

The present invention relates to an apparatus and method for the prediction of a parameter in the blood stream of a subject. The invention is particularly suited, but not limited to predicting a level of glycosylated hemoglobin (HbA1c) in an individual.

### BACKGROUND TO THE INVENTION

The following discussion of the background of the invention is intended to facilitate an understanding of the present invention. However, it should be appreciated that the discussion is not an acknowledgement or admission that any of the material referred to was published, known or part of the common general knowledge in any jurisdiction as at the priority date of the application.

Red blood cells in a blood stream of an individual contain hemoglobin which combines with glucose in the blood to form glycosylated hemoglobin (HbA1c). The reaction of combining glucose with hemoglobin generally occurs over a 10 week period. There is a correlation between glucose level and HbA1c. Typically, the higher the glucose level, the higher the percentage of HbA1C in the blood stream. As red blood cells typically live for 8 -12 weeks before they are replaced, measuring the HbA1c level in the blood stream provides an indication of the level of glucose in the individual's body. More importantly, the “precise degree” of control in an individual's blood glucose over the past 8-12 weeks may be predicted, which is independent and distinct from the spot level of glucose at any point of time.

Typically, in humans, a normal non-diabetic person's. HbA1C level is 3.5-5.5%. For diabetic subjects, a HbA1c level of 6.5% is still considered to be under control. If the subject's HbA1c level is about 7.0%, it denotes suboptimal control and 8.0% is unacceptable.

In addition to providing an indication of glucose in the blood stream of a subject, the prediction and control of HbA1C level also strongly co-relates to outcome in strokes, heart attacks and renal failure resulting from illnesses such as diabetes.

HbA1c has been set as treatment target in many countries, and the level of the same monitored to provide an indication of whether a subject's glucose level is properly under control. However, monitoring is generally by means of invasive analysis where blood samples are taken from an individual.

There is currently no comprehensive suite for the non-invasive measurement and prediction of HbA1c level in a subject, and further there is no apparatus which could predict the HbA1c level in a subject without some form of Calibration required between each individual subject. In particular, there is a need for more predictable tests that what is known for the diagnosis of illnesses such as Diabetes mellitus which is becoming an increasingly common problem.

The present invention provides a reliable invasive method for analysis the parameters in an individual's blood and further alleviates many of the drawbacks of the prior art.

### SUMMARY OF THE INVENTION

Throughout this document, unless otherwise indicated to the contrary, the phrase “comprising”, “consisting of”, and the like, are to be construed as inclusive and not exhaustive.

In accordance with a first aspect of the invention there is an apparatus for predicting a parameter in the blood stream of a subject comprising a laser diode source arranged to emit light of at least two different wavelengths; a first optical receiver arranged to receive incident light of the two different wavelengths where the subject is not present; a second optical receiver arranged to receive transmitted or diffuse reflected light of the two different wavelength when a desired part of the subject is present; and a processor for calculating the ratio of the intensity of the received transmitted or diffuse reflected light to incident light for each of the at least two different wavelengths to provide an indication of the parameter in the blood stream of the subject.

Where the parameter to be predicted is the level of glycosylated hemoglobin (HbA1c), the indication of the parameter in the blood stream of the subject is calculated according to the following formula where there are exactly two wavelengths present:

$$R = \frac{- {α}_{1 Hb} ln ( \frac{{I}_{2}}{{I}_{0 \; 2}} ) + {α}_{2 Hb} ln ( \frac{{I}_{1}}{{I}_{01}} )}{ln ( \frac{{I}_{1}}{{I}_{01}} ) ( {α}_{2 Hb} - {α}_{2 HbA 1 c} ) - ln ( \frac{{I}_{2}}{{I}_{02}} ) ( {α}_{1 Hb} - {α}_{1 HbA 1 c} )}$$

where α_{1HbA1c}, α_{2HbA1c}, α_{1Hb} and α_{2Hb} are the extinction coefficient of HbA1c and the extinction coefficient of ordinary hemoglobin (Hb) at the two selected wavelengths subscripted 1 and 2 respectively; and

$$\frac{{I}_{1}}{{I}_{01}} , \frac{{I}_{2}}{{I}_{02}}$$

are the ratios of the intensity of the received transmitted light or diffuse reflected light to incident light for each of the exactly two different wavelengths.

Preferably, one of the at least two different wavelengths is between 1650 to 1660 nanometers and another of the at least two different wavelength is between 1680 to 1700 nanometers.

Preferably the first optical receiver comprises an optical lens pair and the second optical receiver comprises an optical probe.

In accordance with a second aspect of the invention there is a optical probe for use in the apparatus for predicting a parameter in the blood stream of a subject, the optical probe comprises an input fiber and a plurality of collection fibers; wherein the distance between each of the plurality of collection fibers and the input fiber is between 0.5 millimeters to 2 millimeters.

Preferably the optical probe is disc shaped with the input fiber at the centre and the collection fibers disposed in the circumference of the optical probe.

In accordance with a third aspect of the invention there is a method for predicting a parameter in the blood stream of a subject comprising the following steps: a. emitting at least two different light wavelengths from the laser diode source; b. receiving incident light of the two different light wavelengths from a first optical receiver where the subject is not present; c. receiving transmitted light or diffuse reflected light of the two different light wavelength from a second optical receiver when a desired part of the subject is present; d. calculating the ratio of the intensity of the received transmitted or diffuse reflected light .to incident light for each of the at least two different wavelengths to provide an indication of the parameter in the blood stream of the subject.

Where the parameter to be predicted is the level of glycosylated hemoglobin (HbA1c), the indication of the parameter in the blood stream is calculated according to the following formula where there are exactly two wavelengths present:

$$R = \frac{- {α}_{1 Hb} ln ( \frac{{I}_{2}}{{I}_{0 \; 2}} ) + {α}_{2 Hb} ln ( \frac{{I}_{1}}{{I}_{01}} )}{ln ( \frac{{I}_{1}}{{I}_{01}} ) ( {α}_{2 Hb} - {α}_{2 HbA 1 c} ) - ln ( \frac{{I}_{2}}{{I}_{02}} ) ( {α}_{1 Hb} - {α}_{1 HbA 1 c} )}$$

where α_{1HbA1c}, α_{2HbA1c}, α_{1Hb} and α_{2Hb} are the extinction coefficient of HbA1c and the extinction coefficient of ordinary hemoglobin (Hb) at the two selected wavelengths subscripted 1 and 2 respectively; and

$$\frac{{I}_{1}}{{I}_{01}} , \frac{{I}_{2}}{{I}_{02}}$$

are the ratios of the intensity of the received transmitted or diffuse reflected light to incident light for each of the two different wavelengths.

Preferably, one of the at least two different wavelengths is between 1650 to 1660 nanometers and another one of the at least two different wavelength is between 1680 to 1700 nanometers.

Preferably, the first optical receiver comprises an optical lens pair and the second optical receiver comprises an optical probe.

### BRIEF DESCRIPTION OF THE DRAWINGS

The following invention will be described, by way of example only, with reference to the following drawings of which:

FIG. 1 presents comparison between an individual whose HbA1c level is not properly controlled (FIG. 1a) versus one that is properly controlled (FIG. 1b) over a defined period.

FIG. 2. presents a setup for obtaining HbA1c according to an embodiment of the invention.

FIG. 3 is a table showing the relationship between HbA1c (in percentage) with the corresponding average blood glucose level (mmol/L).

FIG. 4 shows an HbA1c spectrum obtained from a FTIR. spectroscopy in the near infra-red range for identifying the infra-red wavelengths for use in the algorithm according to an embodiment of the invention.

FIG. 5a and FIG. 5b are plots showing the relationship between the percentage of HbA1c and the intensity of absorption of the specified infra-red wavelengths at 1650 nm and 1690 nm respectively.

FIG. 6 is a plot of the predicted percentage HbA1c obtained from the algorithm according to an embodiment against the real value (from a human sample HbA1c solution).

FIG. 7 is a table depicting values of predicted percentage of HbA1c obtained from the algorithm against the real value with varying infra-red wavelengths as that used in FIG. 6.

FIG. 8 presents a detailed layout of the optical probe as presented in FIG. 2.

FIG. 9 presents a plot of the predicted percentage HbA1c levels (using the algorithm) for the six test subjects against a reference percentage HbA1c level obtained via Bayer's invasive method.

FIG. 10a presents a plot of the predicted percentage HbA1c levels (using the algorithm) for the ten test subjects against a reference percentage HbA1c level obtained via a clinical trial.

FIG. 10b presents a plot of the predicted percentage HbA1c levels (using Bayer's invasive method) for the ten test subjects against a reference percentage HbA1c level obtained via a clinical trial.

### DETAILED DESCRIPTION OF THE EMBODIMENTS

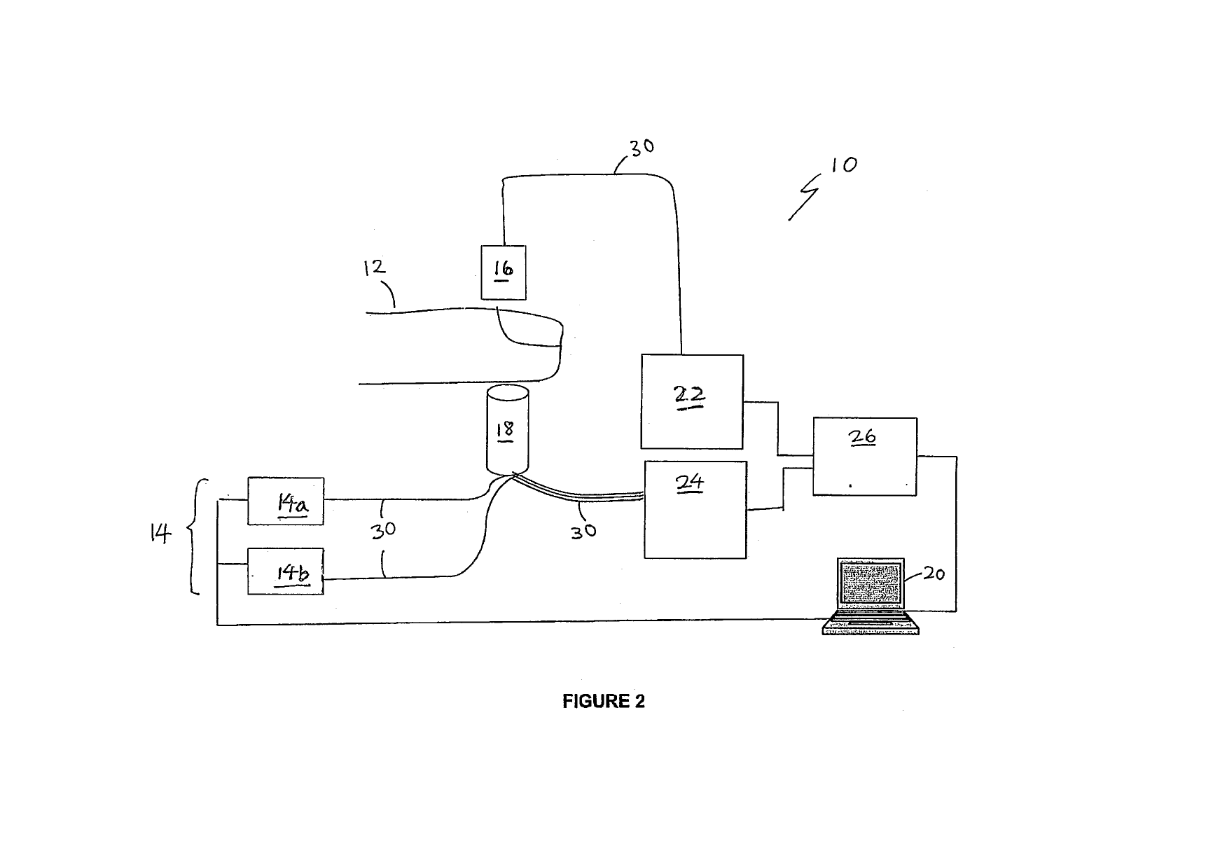

In accordance with an embodiment of the invention there is an apparatus 10 for predicting a parameter in the blood stream of a subject 12 comprising a laser diode source 14; a first optical receiver 16; a second optical receiver 18; and a processor 20 as shown in FIG. 2.

Laser diode source 14 comprises two laser diodes 14a, 14b. Each laser diode 14a, 14b is in data communication with the processor 20. Each laser diode 14a, 14b is controlled by the processor 20 to produce infra-red radiation of a specific wavelength.

The first optical receiver 16 is an optical lens pair and the second optical receiver 18 is an optical probe. The first optical receiver 16 and the second optical receiver 18 are spaced apart such that a desired part of the subject 12, in this case a finger, could be inserted therebetween. It is to be appreciated that other suitable parts of a subject 12 may be used, such as for example, toes.

The first optical receiver 16 is connected via optical fiber 30 to a photo detector 22. The second optical receiver 18 is connected via optical fiber 30 to another photo detector 24. Both photo detectors 22, 24 are in data communication with a database 26 which is coupled with processor 20.

In use, the first optical receiver 16 is arranged to receive incident light of the two different light wavelengths where the subject 12 is not present. The second optical receiver 18 is arranged to receive transmitted or diffuse reflected light of the two different wavelengths when a finger of subject 12 is present.

The above apparatus 10 is suited to measure the level of glycosylated hemoglobin (i.e. HbA1c) of a subject 12 as follows and is subsequently described in this context. In particular, the choice of near infra-red light wavelengths for the laser diodes 14, the design of the optical probe 18 and an algorithm for calculating the HbA1c are described below.

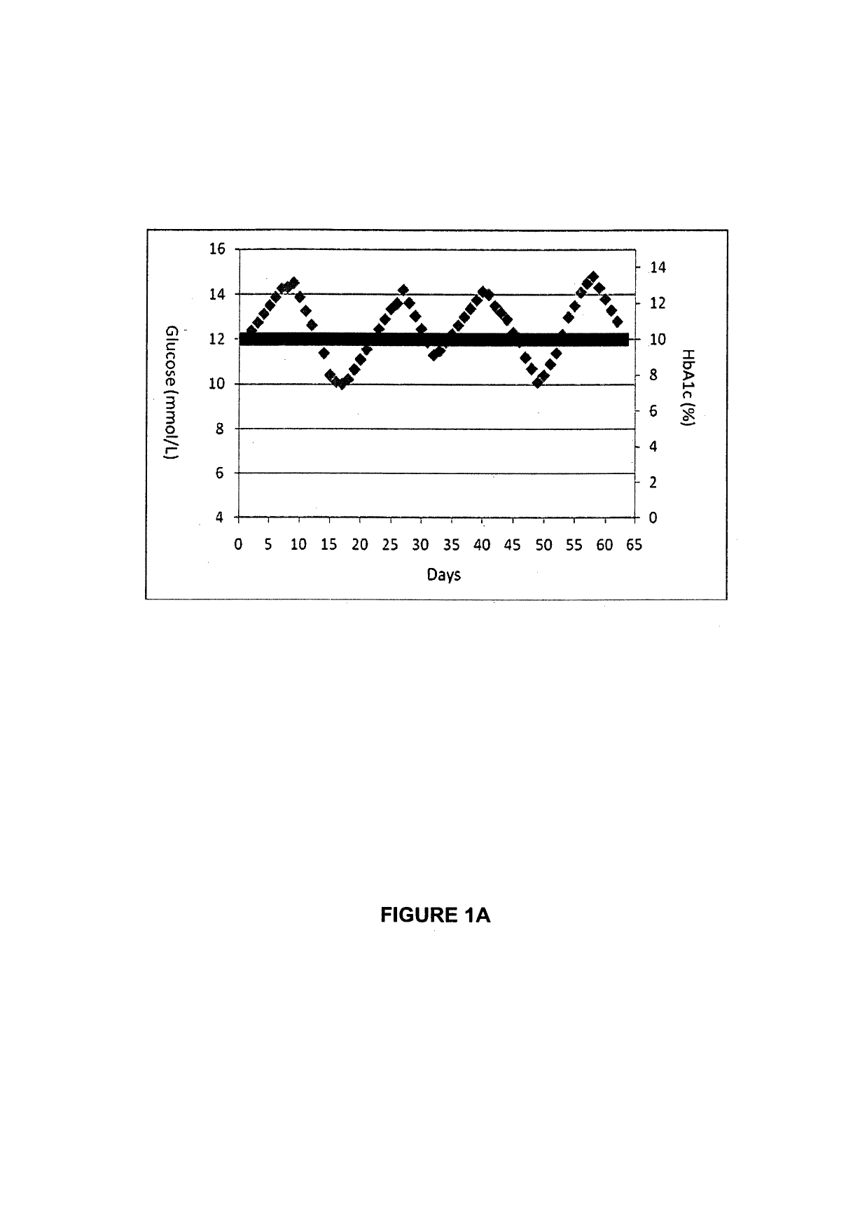

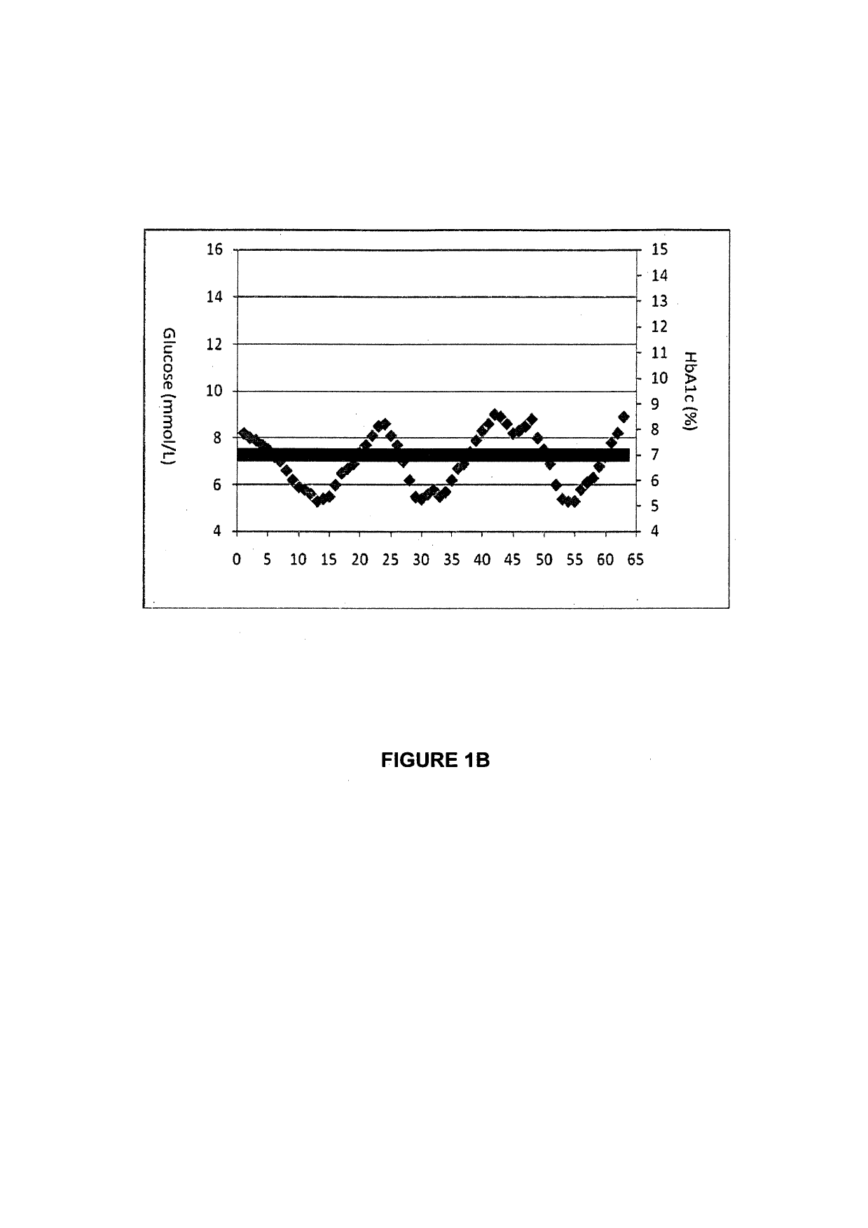

To show how parameters in blood can vary, FIG. 1a shows a graph of glucose changes over 9 weeks for a subject whose HbA1c is not properly controlled. The glucose changes between 10 to 15 mmol/L. This results in an average HbA1c level of 10% at the end of the 9 weeks (solid line), which is above the benchmark of 7%.

In contrast, FIG. 1b shows a graph of glucose changes over the same 9 weeks for a subject whose HbA1c is properly controlled. The glucose changes between 5 to 9 mmol/L. This results in an average HbA1c level of 7% at the end of the 9 weeks (which is within acceptable range).

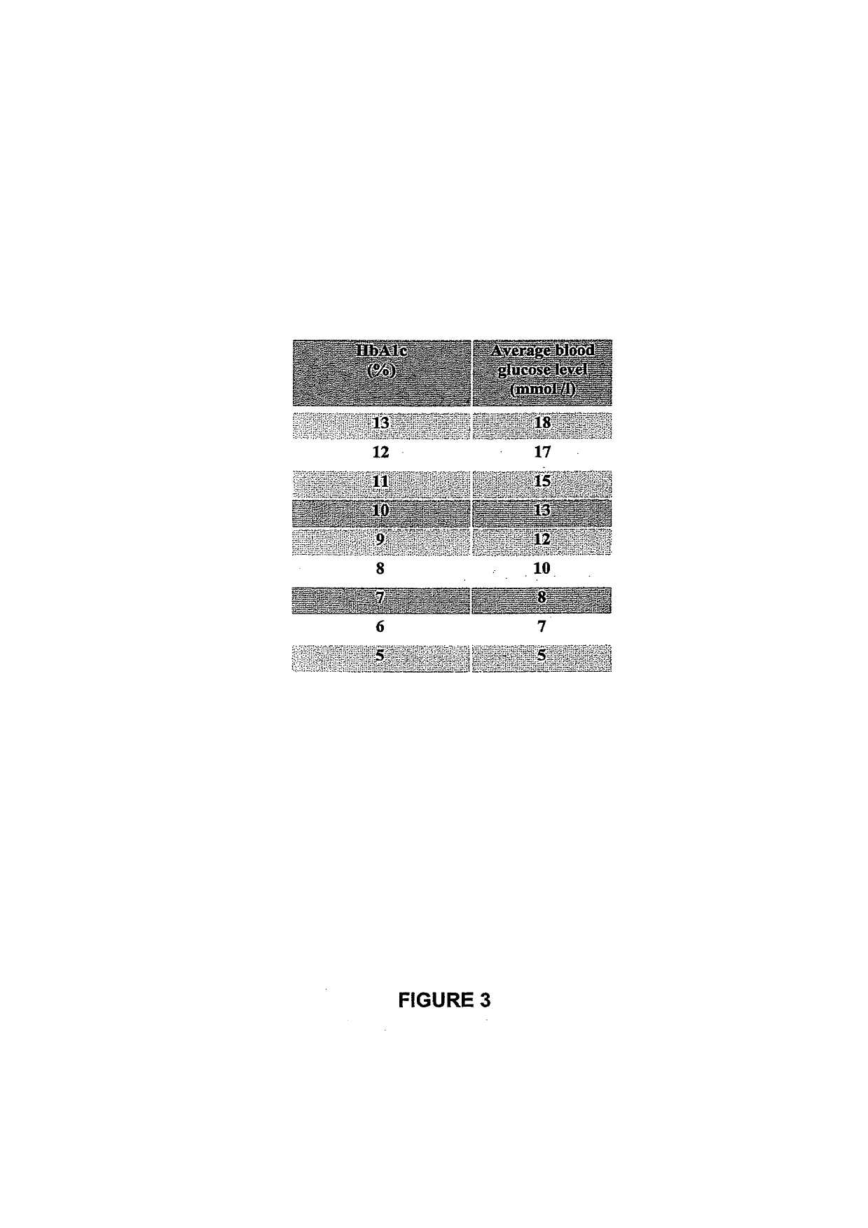

The Applicant discovered that the level of HbA1c in a person is nearly always equal to the glucose level. As shown in FIG. 3, an HbA1c level of 10% correlates to an average glucose level of 13mmol/l. At lower levels there is a smaller difference, so an HbA1c level of 7% meant that the average glucose level was 8 mmol/L.

An in-vitro investigation was setup based on the following control parameters:
Using a human sample (0.115-0.23 mmol/L) of HbA1c analyzed using Fourier Transform infra-red (FTIR) Spectrometer, where the infra red wavelength used is between 1000 to 2500 nanometers.

The in-vitro investigation was setup for the purpose of identifying the absorption peak and trough of HbA1c based on the human sample.

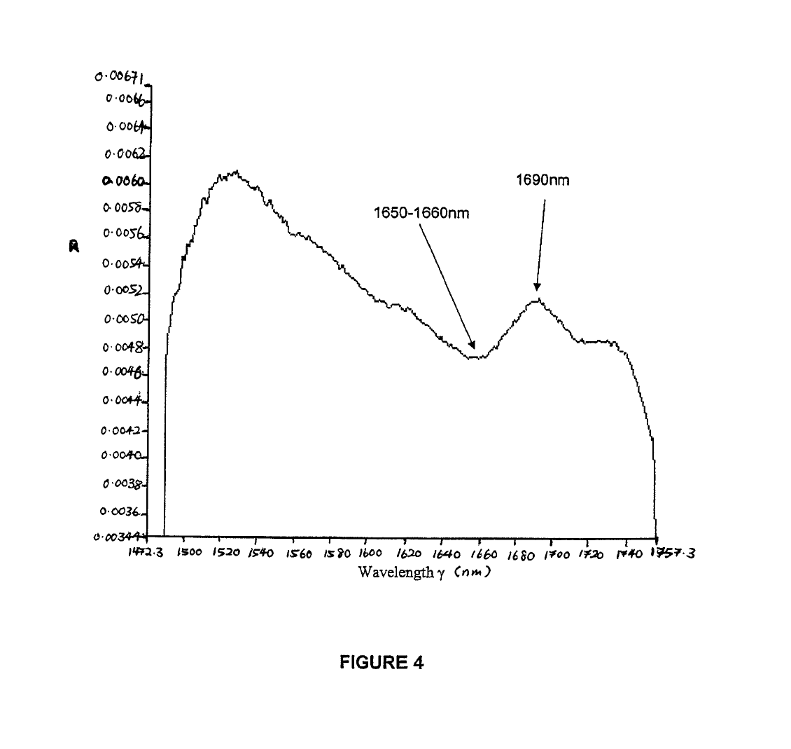

From the FTIR spectrometer, the HbA1c spectrum in the near infra-red NIR range (as shown in FIG. 4) was obtained. From the spectrum presented in FIG. 4, the absorption peak of HbA1c is identified to be at the wavelength of 1690 nm ±10 nm; and the absorption trough is identified to be at the wavelength of between 1650 nm to 1660 nm.

Upon identifying the absorption peak and absorption trough from the FTIR spectrometer, laser diode source 14 is programmed to emit infra-red wavelengths of 1650 and 1690 nanometers for subsequent trials. Specifically, laser diode 14a is controlled by processor 20 to produce an infra-red radiation wavelength of between 1650 to 1660 nanometers and laser diode 14b is controlled to produce a wavelength between 1680 to 1700 nanometers.

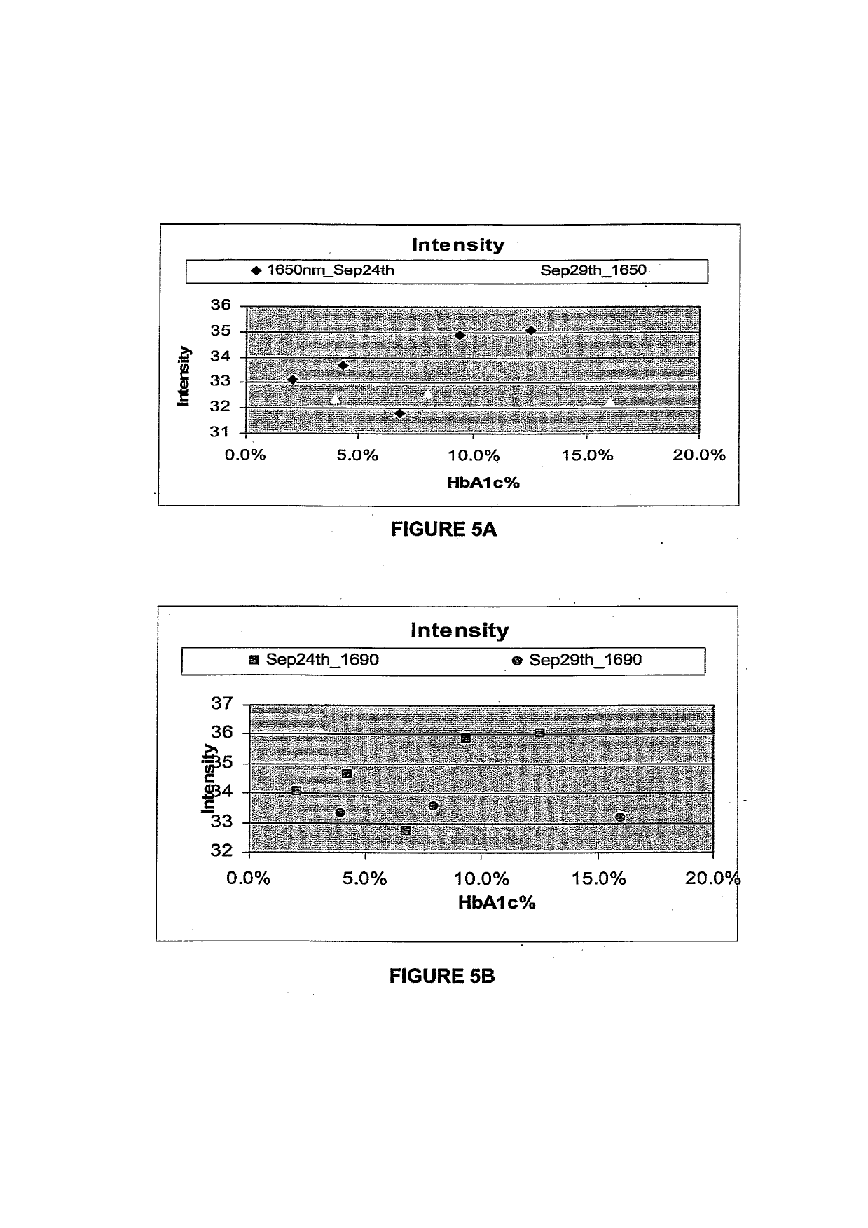

Based on the in-vitro investigation mentioned above, at the specified infra-red wavelengths of 1650 nm (absorption trough) and 1690 nm (absorption peak), there was no obvious trend or co-relationship noted between the percentage of HbA1c and the intensity of infra-red wavelength absorption for each laser diode (see FIG. 5a—for laser diode 14a and FIG. 5b for laser diode 14b). There is thus a need to derive an algorithm or equation for purposes of correlating the intensity of the infra-red wavelength and the Hbn1c value. It is also necessary for the algorithm to be undergo subsequent trials, which seek to:
(i.) obtain the extinction coefficients of HbA1c and hemoglobin (Hb) at each infra-red wavelength;
(ii.) verify an algorithm for calculating ratio of HbA1c to (Hb+HbA1c); and
(iii.) predict the percentage ratio of HbA1c/(Hb+HbA1c) on test subjects 12.

The algorithm is developed to relate the intensity of the selected wavelengths of the laser diodes and the percentage changes of HbA1c. The algorithm is derived based on the principle of calculating ratios of the intensity of the received transmitted or diffuse reflected light at photo detector 24 to incident light at photo detector 22 for each of the at least two different wavelengths from laser diode 14a, 14b.

The algorithm in the form of an equation (1) is presented as follows:

$$R = \frac{- {α}_{1 Hb} ln ( \frac{{I}_{2}}{{I}_{0 \; 2}} ) + {α}_{2 Hb} ln ( \frac{{I}_{1}}{{I}_{01}} )}{ln ( \frac{{I}_{1}}{{I}_{01}} ) ( {α}_{2 Hb} - {α}_{2 HbA 1 c} ) - ln ( \frac{{I}_{2}}{{I}_{02}} ) ( {α}_{1 Hb} - {α}_{1 HbA 1 c} )} \qquad ( 1 )$$

Where R is the ratio of HbA1c concentration and total hemoglobin concentration (ordinary hemoglobin+HbA1c);

α_{1HbA1c}, α_{1Hb}, α_{2HA1c}, and α_{2Hb} are the extinction coefficient of HbA1c, extinction coefficient of ordinary hemoglobin at two selected wavelengths (subscripted 1 and 2 respectively, where subscript 1 corresponds to the first wavelength and subscript 2 corresponds to the second wavelength). These coefficients are obtained via experiment; and

I₁, I₀₁, I₂, and I₀₂ are transmitted light intensity and incident light intensity at two selected wavelengths (subscripted 1 and 2).

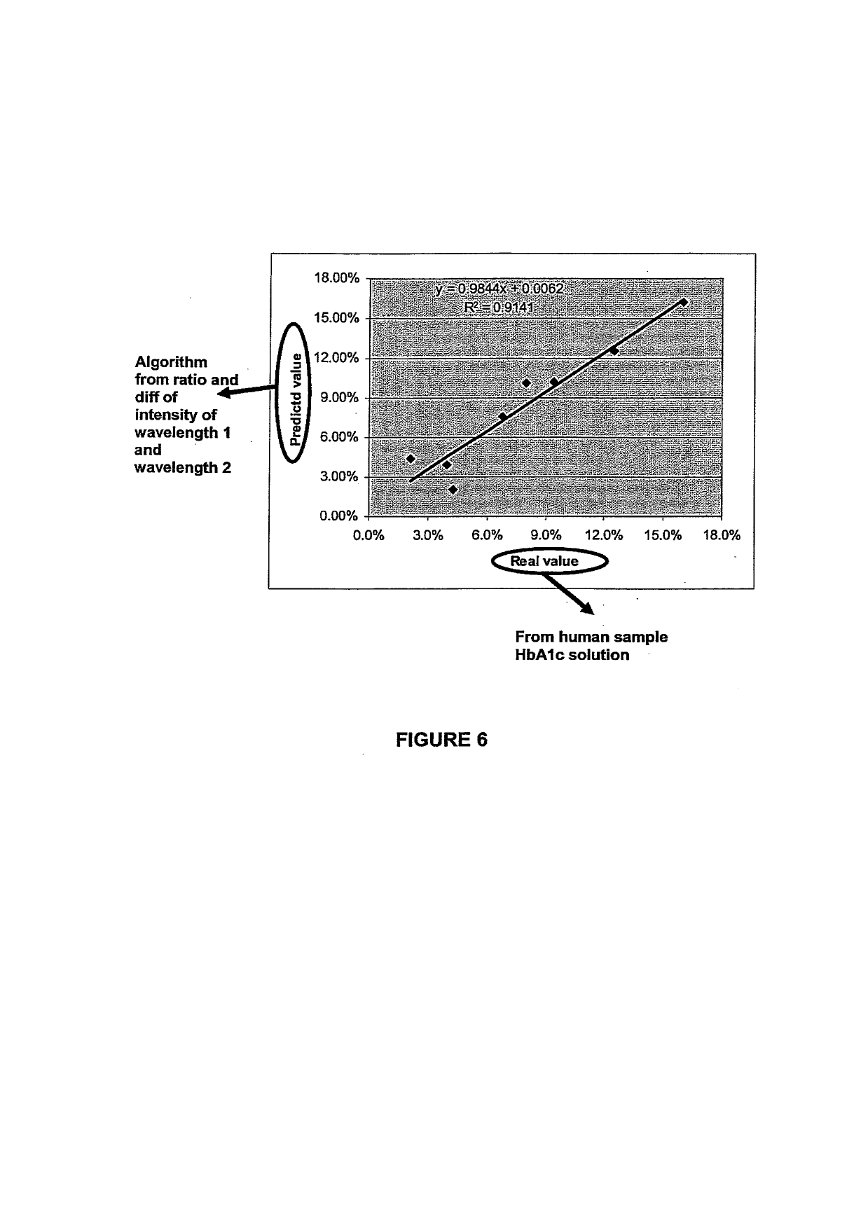

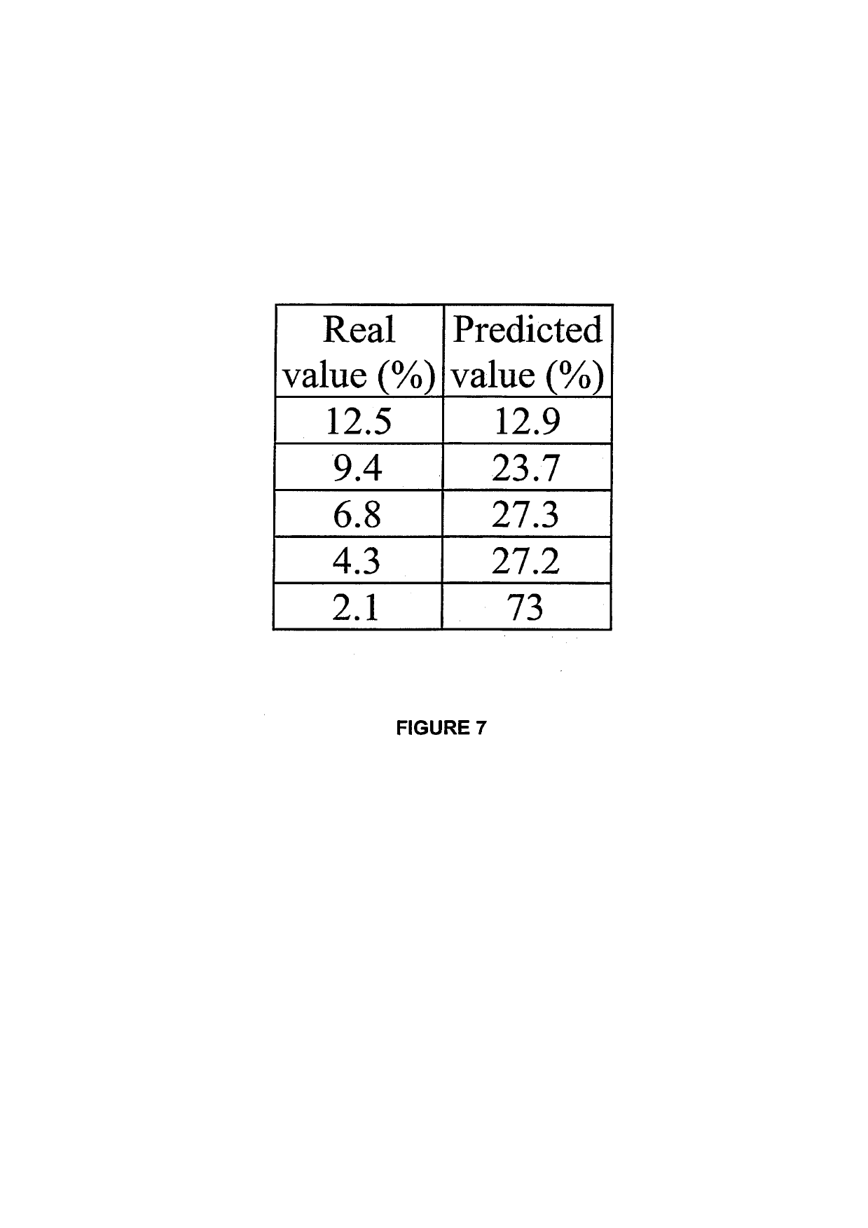

Using the algorithm, an almost linear relationship between the predicted value (algorithm) and real value (from human sample HbA1c solution) is obtained (see FIG. 6). However, it is to be noted that if other wavelengths were chosen (e.g. 1690 nm and 1732 nm) the HbA1c values would not be predicted as they are not out of the peak absorption wavelength of 1690 nm. In FIG. 7, a real value of 6.8% of HbA1c corresponds to a predicted value of 27.3%, which is way off mark.

Upon successfully obtaining a linear corresponding relationship, the apparatus 10 as shown in FIG. 2 is prepared for non-invasive measurement of test subjects 12. Before the subject 12 finger is positioned between the optical lens 14 and optical probe 16, I₀₁ and I₀₂ are acquired via photo detector 22. When a test subject's finger is positioned between the optical lens and optical probe (as seen in FIG. 2), I₁ and I₂ are acquired via photo detector 24 while the finger is on the optical probe. The laser diodes having the two identified infra-red wavelengths (as discussed earlier) are controlled by the processor 20 with a data acquisition system which is synchronized to the laser diodes 14a, 14b.

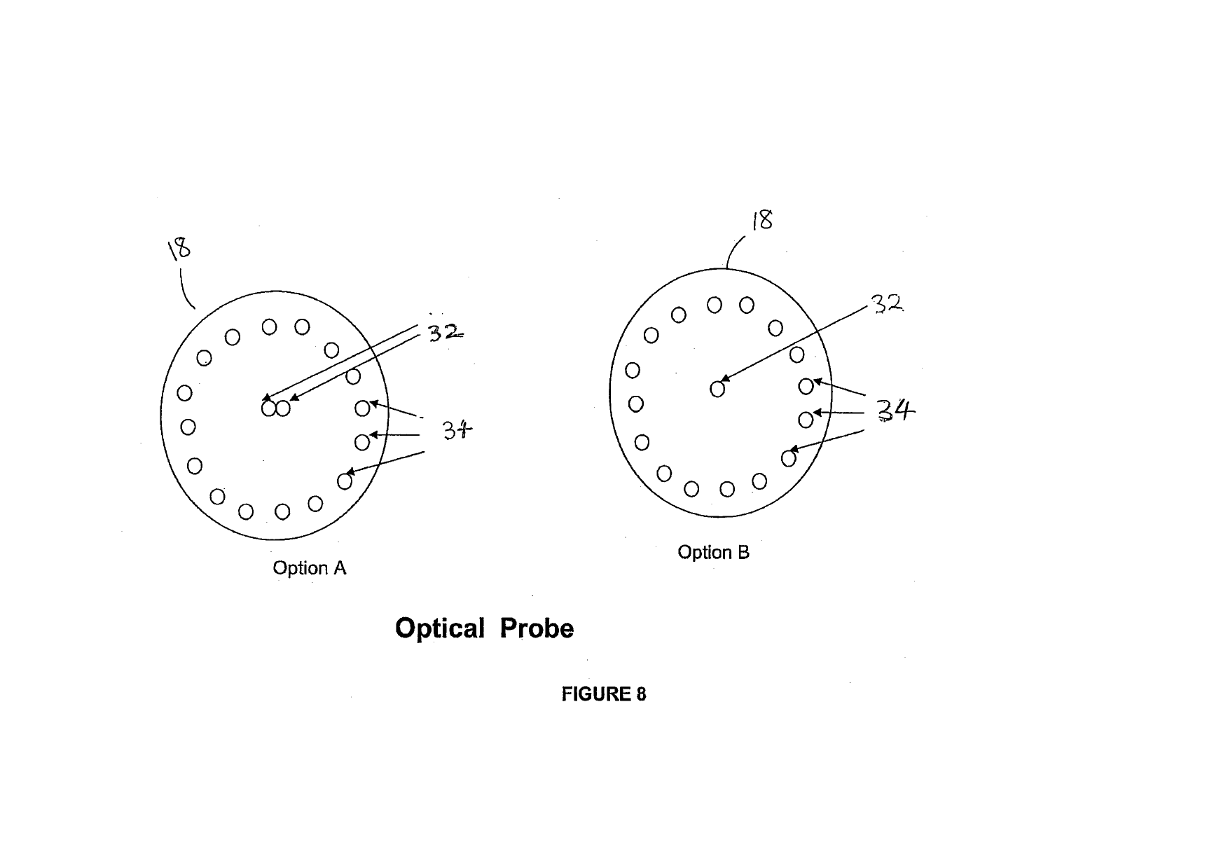

It is to be noted that care has to be taken to ensure that the optical probe 18 design is properly achieved. As seen in FIG. 8, two options are provided for the design of the optical probe 18. The first option (Option A) provides a configuration where there is a separate optic fiber for laser diode 14a and laser diode 14b. The second option (Option B) envisage the optic fibers for laser diode 14a and laser diode 14b being coupled together using a fiber coupler. In both options, care must be taken to ensure that the distance between the input fiber 32 to output fiber 34 is 0.5 millimeters to 2 millimeters for maximization (optimization) of signals.

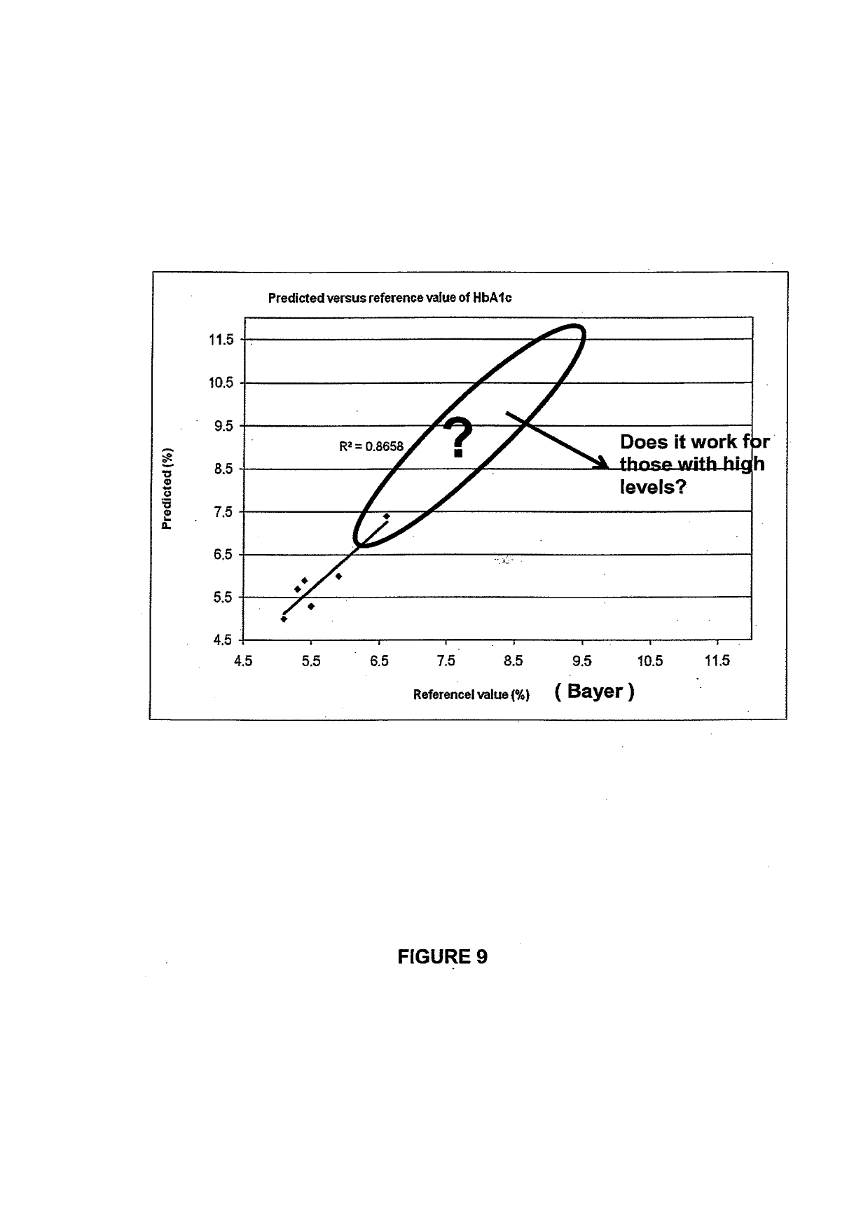

Using the apparatus 10, a first trial was carried out with six test subjects 12. The test subjects 12 are normal individuals with low HbA1c levels (i.e. non-diabetic). The predicted percentage HbA1c levels for the six test subjects is plotted against a reference percentage HbA1c level, preferably obtained via Bayer's invasive method which is well known. An approximately linear relationship is obtained as shown in FIG. 9.

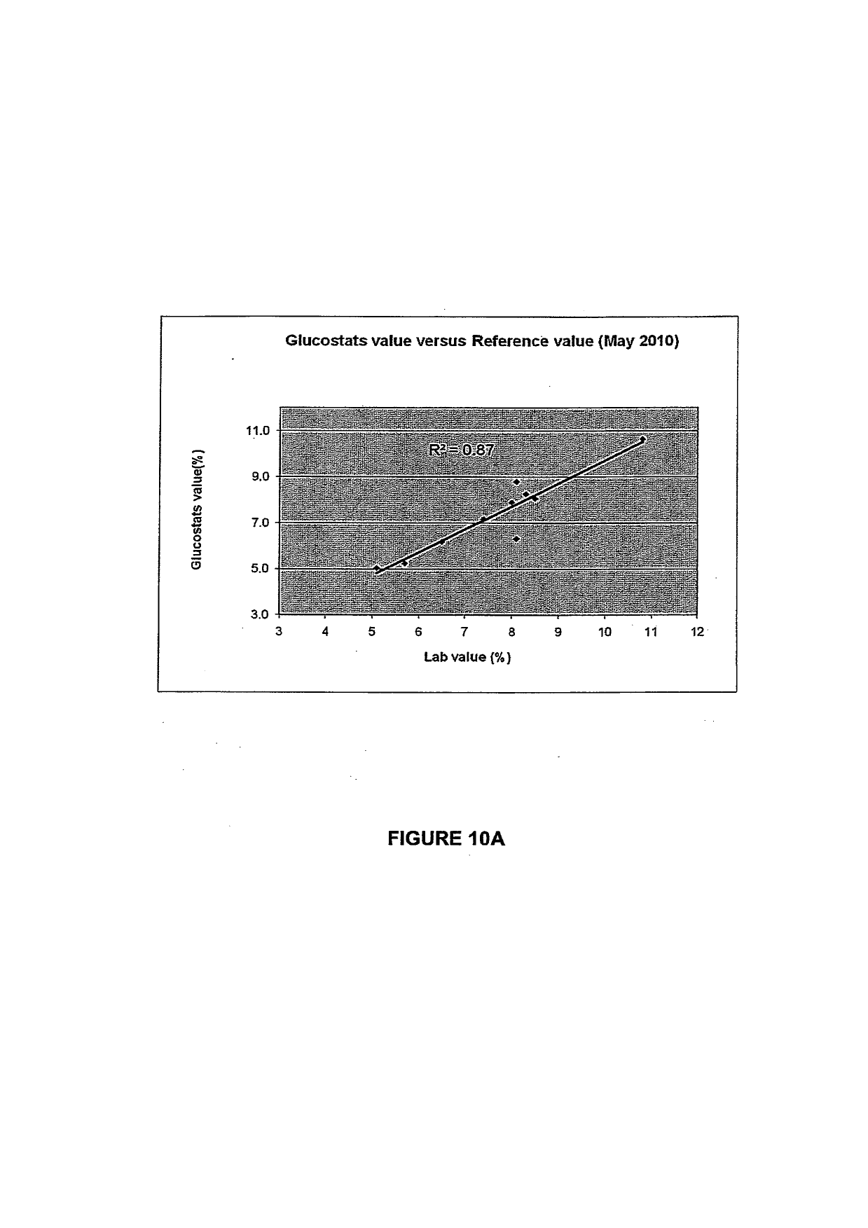

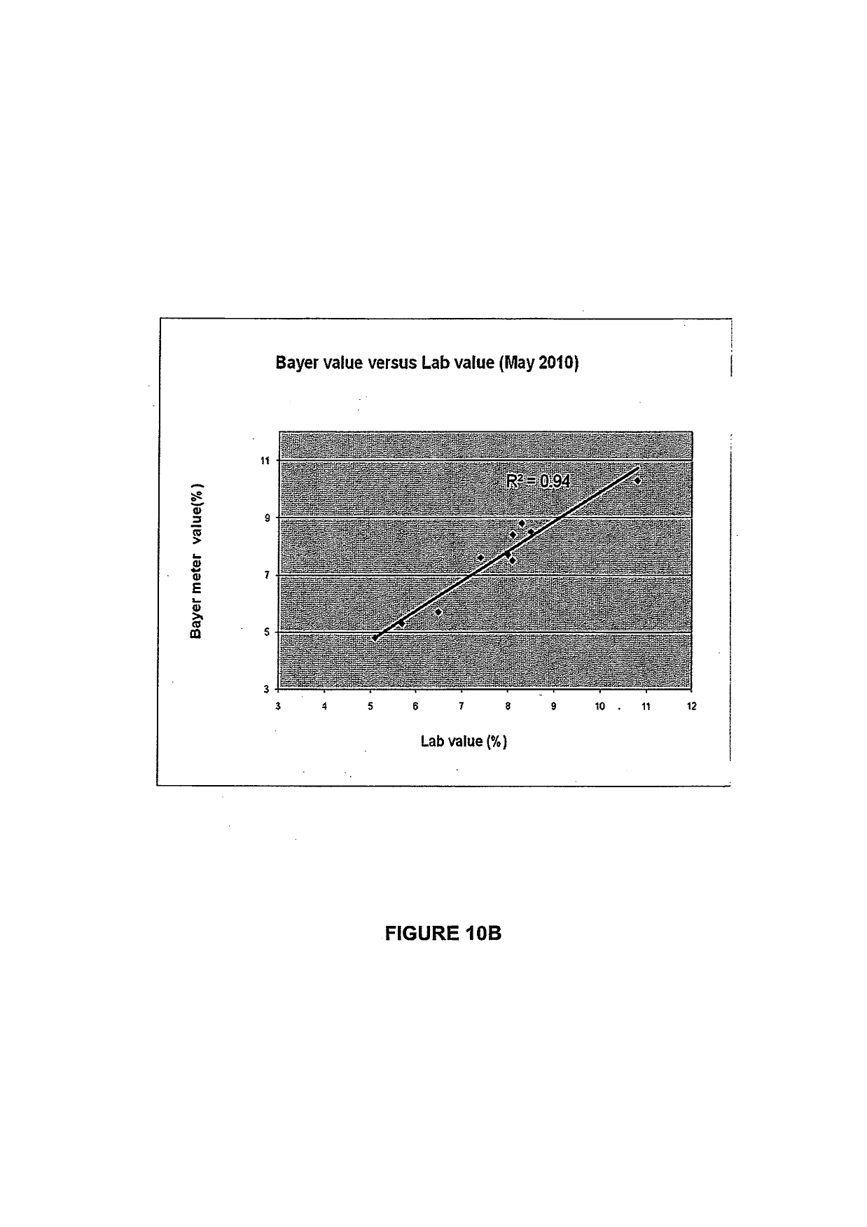

The apparatus 10 is then further performed for ten individuals with high level of HbA1c or poorly controlled diabetes mellitus. A clinical trial is performed, with laboratory results obtained. These laboratory results were compared with the predicted values obtained from the algorithm as presented in equation (1)—see FIG. 10a, as well as with the Bayer's invasive method—see FIG. 10b.

Based on the results obtained from the algorithm, there is a strong linear co-relation of R>0.9 (i.e. R²=0.874→R=0.93)

It is to be appreciated that the invention is focused on the combination of algorithm and the choice of two specific wavelengths to yield HbA1c prediction.

The two specific wavelengths may be chosen from a range of 1650 to 1660 nm for the trough wavelength and 1680 to 1700 nm for the peak wavelength.

It is to be further appreciated that according to the algorithm of formula (1), any two wavelengths at absorption peak and trough can be used to calculate the percentage HbA1c, however, wavelengths of 1650 nm and 1690 nm are chosen because the laser diodes at the two wavelengths are available.

It is to be appreciated that the above described steps of locating peak and trough absorption rates from a FTIR spectrum; in-vitro trials for determining correlation between the intensity of infra-red wavelength absorption and the percentage of HbA1c may be generalized to other parameters such as glucose in the blood stream other than HbA1c, as different parameters have their own set of peak/trough absorption rates and extinction coefficients.

The invention utilizes the correlation between multiple peaks in the spectrum derived of the FTIR (e.g. in FIG. 4). As such, the minimum number of wavelengths required is two (peak, trough). More wavelengths, however, may be added to the algorithm in equation (1). In such instances, further extinction coefficients for each infra-red wavelengths need to be determined and added (or subtracted) to the equation (1).

It should be further appreciated by the person skilled in the art that features and modifications discussed above, not being alternatives or substitutes, can be combined to form yet other embodiments that fall within the scope of the invention described.

1-15. (canceled)

16. An apparatus for predicting a parameter in the blood stream of a subject comprising:
a laser diode source arranged to emit light of at least two different wavelengths;
a first optical receiver arranged to receive incident light of the at least two different wavelengths where the subject is not present;
a second optical receiver arranged to receive transmitted light of the at least two different wavelengths when a desired part of the subject is present; and
a processor for calculating the ratio of the intensity of the received transmitted or diffuse reflected light to incident light for each of the at least two different wavelengths to provide an indication of the parameter in the blood stream of the subject.

17. The apparatus according to claim 16, wherein the at least two different light wavelengths are infra-red wavelengths selected by identifying an absorption peak and an absorption trough on a Fourier Transform infra-red (FTIR) spectrum obtained in response of the infra-red wavelengths to the parameter.

18. The apparatus according to claim 16, wherein the parameter to be predicted is the level of glycosylated hemoglobin (HbA1c).

19. The apparatus according to claim 18, wherein the indication of the parameter in the blood stream of the subject is calculated according to the following formula where there are exactly two wavelength present:
$$R = \frac{- {α}_{1 Hb} ln ( \frac{{I}_{2}}{{I}_{0 \; 2}} ) + {α}_{2 Hb} ln ( \frac{{I}_{1}}{{I}_{01}} )}{ln ( \frac{{I}_{1}}{{I}_{01}} ) ( {α}_{2 Hb} - {α}_{2 HbA 1 c} ) - ln ( \frac{{I}_{2}}{{I}_{02}} ) ( {α}_{1 Hb} - {α}_{1 HbA 1 c} )}$$
where α_{HbA1c}, α_{2HbA1c}, α_{1Hb} and α_{2Hb} are the extinction coefficient of HbA1c and the extinction coefficient of ordinary hemoglobin (Hb) at the two selected wavelengths subscripted 1 and 2 respectively; and
$$\frac{{I}_{1}}{{I}_{01}} , \frac{{I}_{2}}{{I}_{02}}$$
are the ratios of the intensity of the received transmitted or diffuse reflected light to incident light for each of the two different wavelengths.

20. The apparatus according to claim 16, wherein one of the at least two different wavelengths is between 1650 to 1660 nanometers and another of the at least two different wavelengths is between 1680 to 1700 nanometers.

21. The apparatus according to claim 16, wherein the first optical receiver comprises an optical lens pair and the second optical receiver comprises an optical probe.

22. An optical probe for use in an apparatus for predicting a parameter in the blood stream of a subject according to claim 20, the optical probe comprising an input fiber and a plurality of collection fibers; wherein the distance between each of the plurality of collection fibers and the input fiber is between 0.5 millimeters to 2 millimeters.

23. The optical probe according to claim 22 wherein the optical probe is disc shaped with the input fiber at the centre and the collection fibers disposed in the circumference of the optical probe.

24. A method for predicting a parameter in the blood stream of a subject comprising the following steps:
a. emitting at least two different light wavelengths from a laser diode source;
b. receiving incident light of the at least two different light wavelengths from a first optical receiver where the subject is not present;
c. receiving transmitted or diffuse reflected light of the at least two different light wavelengths from a second optical receiver when a desired part of the subject is present; and
d. calculating the ratio of the intensity of the received transmitted or diffuse reflected light to incident light for each of the at least two different wavelengths to provide an indication of the parameter in the blood stream of the subject.

25. The method according to claim 24, wherein the at least two different light wavelengths are infra-red wavelengths selected by identifying an absorption peak and an absorption trough on a Fourier Transform infra-red (FTIR) spectrum obtained in response of the infra-red wavelengths to the parameter.

26. The method according to claim 24, wherein the parameter to be predicted is the level of glycosylated hemoglobin (HbA1c).

27. The method according to claim 26, wherein the indication of the parameter in the blood stream is calculated according to the following formula where there are exactly two wavelengths present:
$$R = \frac{- {α}_{1 Hb} ln ( \frac{{I}_{2}}{{I}_{0 \; 2}} ) + {α}_{2 Hb} ln ( \frac{{I}_{1}}{{I}_{01}} )}{ln ( \frac{{I}_{1}}{{I}_{01}} ) ( {α}_{2 Hb} - {α}_{2 HbA 1 c} ) - ln ( \frac{{I}_{2}}{{I}_{02}} ) ( {α}_{1 Hb} - {α}_{1 HbA 1 c} )}$$
where α_{HbA1c}, α_{2HbA1c}, α_{1Hb} and α_{2Hb} are the extinction coefficient of HbA1c and the extinction coefficient of ordinary hemoglobin (Hb) at the two selected wavelengths subscripted 1 and 2 respectively; and
$$\frac{{I}_{1}}{{I}_{01}} , \frac{{I}_{2}}{{I}_{02}}$$
are the ratios of the intensity of the received transmitted or diffuse reflected light to incident light for each of the two different wavelengths.

28. The method according to claim 24, wherein one of the at least two different wavelengths is between 1650 to 1660 nanometers and another one of the at least two different wavelengths is between 1680 to 1700 nanometers.

29. The method according to claim 24, wherein the first optical receiver comprises an optical lens pair and the second optical receiver comprises an optical probe.

30. A kit for predicting a parameter in the blood stream of a subject comprising:
an apparatus comprising: a laser diode source arranged to emit light of at least two different wavelengths; a first optical receiver arranged to receive incident light of the at least two different wavelengths where the subject is not present; a second optical receiver arranged to receive transmitted light of the at least two different wavelengths when a desired part of the subject is present; and a processor for calculating the ratio of the intensity of the received transmitted or diffuse reflected light to incident light for each of the at least two different wavelengths to provide an indication of the parameter in the blood stream of the subject; and
a set of instructions for using the apparatus according to a method for predicting a parameter in the blood stream of a subject comprising the following steps:
a. emitting at least two different light wavelengths from a laser diode source;
b. receiving incident light of the at least two different light wavelengths from a first optical receiver where the subject is not present;
c. receiving transmitted or diffuse reflected light of the at least two different light wavelengths from a second optical receiver when a desired part of the subject is present; and
d. calculating the ratio of the intensity of the received transmitted or diffuse reflected light to incident light for each of the at least two different wavelengths to provide an indication of the parameter in the blood stream of the subject.
